(19) United States
(12) Patent Application Publication
(10) Pub. No.: US 2008/0220051 A1
(43) Pub. Date: Sep. 11, 2008

(54) Powdery Sterol Compositions Containing Colloid-Forming Agents

(76) Inventors: Peter Horlacher, Bellenberg (DE); Robert Salacz, Senden (DE); Jorg Schwarzer, Hilden (DE)

Correspondence Address: COGNIS CORPORATION;PATENT DEPARTMENT, 300 BROOKSIDE AVENUE, AMBLER, PA, 19002, US

(21) Appl. No.: 12/064,658

(22) PCT Filed: Aug. 12, 2006
(86) PCT No.: PCT/EP2006/007998

§ 371 Date: Feb. 25, 2008

(30) Foreign Application Priority Data
Aug. 23, 2005 (DE) 10 2005 039 835.9

(51) Int. Cl. A61K 9/14; A61P 9/00

(52) U.S. Cl. 424/439; 424/489

(57) ABSTRACT

Compositions in powder form of sterols, stanols, and esters thereof, and natural macromolecular colloid formers are disclosed. A process of making the compositions is also disclosed. The suitability of incorporation of the compositions in powder form in food products, for example beverages and milk products, is also disclosed.

### CROSS-REFERENCE TO RELATED APPLICATIONS

This application claims priority under 35 U.S.C. Section 119 of International application No. PCT/EP2006/007998 filed Aug. 12, 2006, and German application No. DE 10 2005 039 835.9 filed Aug. 23, 2005, which are incorporated herein by reference in their entireties.

### FIELD OF THE INVENTION

This invention relates generally to foods and, more particularly, to compositions containing sterols, stanols, and/or esters thereof, for incorporation in foods, cosmetic and pharmaceutical preparations, to a process for their production and to products, more particularly foods, which contain these compositions.

### BACKGROUND OF THE INVENTION

The addition of sterols and stanols to foods for their cholesterol-lowering properties and the resulting prevention of future diseases, such as atherosclerosis, heart disease and hypertension, has been known for years. Since phytosterols and stanols are insoluble in water and only poorly soluble in fats and oils, the incorporation of these cholesterol-lowering agents in food preparations, cosmetic or pharmaceutical products poses considerable problems. The unfavorable solubility behavior of the substances results not only in poor dispersibility, but also in reduced bioavailability and in unsatisfactory stability of the food preparations. Accordingly, the prior art proposes numerous formulations for solving these problems.

Efforts to increase solubility included the formulation of esters of the sterols, as described, for example, in European patent application EP 1275309 A1, or esters of the stanols, as described in US patent U.S. Pat. No. 5,502,045, which had slightly improved processability through their better solubility, but which also showed different hypocholesterolaemic activity in relation to the free sterols.

Numerous patent applications describe how the availability of sterols can be improved by reducing their particle sizes, mainly by micronization. A process for the production of a sterol dispersion, in which the sterols have a particle size distribution of 0.1 to 30 μm, is disclosed in International patent application WO 03/105611 A2. Unfortunately, prepared dispersions do not have a long shelf life as a result of sedimentation or the risk of contamination.

However, the micronization of the sterol particles is not in itself sufficient to enable good incorporation to be achieved. Although the bioavailability of the finely dispersed particles can be improved by increasing the surface, the micronized particles show poor wetting behavior, readily aggregate and generally float on water-containing surfaces. In many cases, the ground sterol can only be dispersed in a beverage by special methods involving intensive mixing. However, intensive mixers are not normally available to the end user of the food manufacturers.

Accordingly, many manufacturers combine micronization of the sterols with the additional use of emulsifiers. One example of this are the preparations described in European patent EP 0897671 B1 which contain sterols and sterol esters with a particle size of at most 15 μm in a mixture with emulsifiers, the ratio by weight of emulsifier to sterol in the aqueous phase being less than 1:2. Commonly used emulsifiers are monoglycerides and polysorbates (U.S. Pat. No. 6,623,780 B1, U.S. Pat. No. 6,376,482 B2, WO 02/28204 A1). Powder-form sterol ester formulations having a low protein content and also containing mono- and diglycerides as emulsifiers are disclosed in International patent application WO 03/086468 A1. Even though these emulsifiers are distinguished by high compatibility and have been known for some time as food emulsifiers, efforts are being made to reduce the quantity in which such emulsifiers are used or even to avoid them altogether because emulsifiers also influence the bioavailability of other substances present in the foods or can adversely affect the stability of the formulations.

Avoiding emulsifiers was also the goal of the sterol formulations disclosed in European patent EP 1059851 B1 which contain thickeners for better dispersibility. In International patent application WO 98/13023, micronized sterols based on sugars are processed in aqueous dispersions. However, formulations in the form of aqueous suspensions generally show poor storage stability in regard to their physicochemical and microbiological properties. These storage problems can be avoided by the use of dry formulations.

Numerous methods for improving solubility and dispersibility, such as the formulation of dry powders, are described in International patent application WO 99/63841 A1 which proposes PEG, PVP, copolymers, cellulose ethers and esters as carriers. The direct use of food bases as carriers for powdered sterols is also known, cf. EP 1 003 388 B1.

However, the main obstacle to the use of dry powders containing lipophilic ingredients is the problem of dispersibility. Accordingly, other auxiliaries, generally emulsifiers, usually have to be incorporated in such formulations. Readily dispersible sterol-containing powder formulations are described in European patent EP 0 947 197 B1, International patent application WO 03/000075 A1 and in US patent U.S. Pat. No. 5,932,562. Maltodextrin was the carrier in these formulations, but cannot be used without the aid of emulsifiers.

German Patent Application DE 102 53 111 A1 also discloses powder-form phytosterol formulations with a mean particle size of 0.01 to 100 μm which can readily be redispersed in water without the use of emulsifiers. With the aid of protective colloids, more especially modified starch or gum arabic, albeit in combination with plasticizers, such as sucrose, readily dispersible powders can be produced by spray drying. One disadvantage of adding sugars as plasticizers lies in the lack of a neutral taste of the sterol formulations which limits their subsequent use in various foods.

The problem addressed by the present invention was to provide a formulation which would allow for the simple and effective dispersion and incorporation of sterols and stanols in foods while reducing or completely avoiding the use emulsifiers. The sterol formulation would be easy to produce and would be distinguished by high stability in storage and a neutral taste.

### BRIEF SUMMARY OF THE INVENTION

The present invention provides a composition in powder form which comprises:
(a) a sterol and/or stanol, and/or esters thereof; and
(b) a natural macromolecular colloid former selected from the group consisting of gum arabic, gelatine, tragacanth, an alginate, carrageen, guar gum, xanthan and mixtures thereof, in a ratio of a) to b) of about 10:90 to 80:20.

Another aspect of the present invention Is directed to a process for the production of sterol-containing powders which process comprises:
a) dispersing a micronized sterol and/or stanol, and/or esters thereof, in water heated to about 80 to 100° C. by stirring to form a dispersion,
b) adding a natural macromolecular colloid former selected from the group consisting of gum arabic, gelatine, tragacanth, an alginate, carrageen, guar gum, xanthan, and mixtures thereof, to the dispersion in a ratio of a) to b) of about 10:90 to 80:20,
c) homogenizing the dispersion under high pressure; and
d) spray-drying the homogenized dispersion.

Yet another aspect of the invention is a food product, for example, a beverage or milk product which contains about 0.1 to 50% by weight of the composition of the invention.

The compositions according to the invention possess good solubilizing properties, reduced aggregation and agglomeration properties, and improved wettability, which properties enable sterols, stanols, and esters thereof, to be readily dispersed in water- and fat-containing preparations. They have excellent handling behavior as they can be further processed without expensive machinery, and advantageously result in neutral-tasting sterol-containing preparations having excellent long-term stability.

The preparations according to the invention may readily be incorporated in foods, more particularly in milk, milk beverages, whey and yoghurt beverages, margarine, fruit juices, fruit juice mixtures, fruit juice beverages, vegetable juices, carbonated and still beverages, soya milk beverages or high-protein liquid food substitute beverages and fermented milk preparations, yoghurt, drinking yoghurt or cheese preparations, and in cosmetic or pharmaceutical preparations.

The compositions according to the invention may preferably be produced without emulsifiers. Through the absence of emulsifiers, the occurrence of incompatibility reactions with other auxiliaries can be reduced and the storage stability of the pre-formulation can be significantly improved.

The expression “emulsifier-free” shall be understood to mean that the use of conventional food emulsifiers, such as lecithins, monoglycerides, diglycerides, polysorbates, sodium stearyl lactylate, glycerol monostearate, lactic acid esters and polyglycerol esters, can be avoided. Although emulsifiers naturally occurring in foods, such as cholesterol, for example, cannot be avoided, no emulsifier is intended to be added as an auxiliary during the production of the sterol/stanol compositions according to the invention.

### DETAILED DESCRIPTION OF THE INVENTION

Other than in the operating examples, or where otherwise indicated, all numbers expressing quantities of ingredients or reaction conditions used herein are to be understood as modified in all instances by the term “about.”

The use of “and/or” shall be understood that, for example, “A and/or B” shall encompass the following embodiments: “A” or “B” or “A and B.”

Sterols and/or Stanols

Sterols obtained from plants and vegetable raw materials, so-called phytosterols and phytostanols, are used for the purposes of the invention. Known examples are ergosterol, brassica sterol, campesterol, avenasterol, desmosterol, clionasterol, stigmasterol, poriferasterol, chalinosterol, sitosterol and mixtures thereof. Of these, β-sitosterol and campesterol are preferably used. The hydrogenated saturated forms of the sterols, so-called stanols, are also included among the compounds used, β-sitostanol and campestanol being preferred.

Vegetable raw material sources include, inter alia, seeds and oils of soybeans, canola, palm kernels, corn, cocoa, rape, sugar cane, sunflower, olive, cotton, soya, peanut or products from tall oil production.

In addition, esterification products of the sterols and stanols, preferably with saturated and/or unsaturated C₆₋₂₂ and preferably C₁₂₋₁₈ fatty acids are processed, although the invention is not limited to esters of this type. Phenolic acid esters, more particularly derivatives of cinnamic acid, caffeic add and ferulic acid, may also be used.

Naturally occurring esters may be directly obtained from vegetable raw materials or the sterol/stanol esters can be produced by transesterification with other esters. Derivatives obtained by esterification of free sterols or stanols with the corresponding fatty acids may also be used.

However, the compositions according to the invention preferably contain the free, unesterified sterols and stanols because the esterified derivatives show improved solubility and dispersibility in relation to the free sterols and stanols.

The powder-form compositions preferably contain 30 to 50% by weight and, more particularly 35 to 45% by weight sterols, stanols and/or sterol and stanol esters, based on the emulsifier-free powder-form composition.

Accordingly, the present invention is also directed to compositions containing sterol/stanol (ester) formulations having the above-mentioned composition. They are preferably used in beverages and milk products which contain 0.1 to 50% by weight and preferably 1 to 20% by weight of the powder-form preparations, based on the total weight of the foods.

### Natural Macromolecular Colloid Formers

The natural macromolecular colloid formers which are suitable for use in the present invention are selected from the group consisting of gum arabic, gelatine, tragacanth, alginates, carrageen, guar gum and xanthan, and mixtures thereof. The selected colloid formers are distinguished by a low viscosity after swelling in water. Since poorly soluble sterols and/or stanols are preferably used in the formulations, the choice of the carriers used is typically selected from low-viscosity materials. It has been found in tests that high-viscosity materials, such as modified starches and starch derivatives for example, do not produce stable dispersions.

The ratio according to the invention of a) sterols and/or stanols and/or esters thereof to b) natural macromolecular colloid formers of about 10:90 to 80:20 is determined by the choice of the carrier materials. If excessively large amounts of colloid formers are used, the stability of the dispersion decreases and the spraying properties of the dispersion during production of the powder deteriorate, as do the favorable dispersion properties of the final formulation in various foods. If the amounts of carrier materials used are too small, the wettability of the powder and hence its ready incorporation in foods is adversely affected. Accordingly, a ratio of a) to b) of 35:65 to 65:35 is preferred, a ratio of 35:65 to 45:55 being particularly preferred.

In the particularly preferred compositions, the content of other auxiliaries in the sterol-containing powder formulation is minimal, so that the compositions according to the invention preferably contain about 50 to 70% by weight and, more particularly, about 55 to 65% by weight of natural macromolecular colloid formers, based on the powder-form preparation, gum arabic being the most suitable.

### Gum Arabic

Gum arabic is a naturally occurring polysaccharide with an average molecular weight of 260,000 to 1,160,000 daltons which is composed of a mixture of the calcium, magnesium and potassium salts of polyarabic acid. The structural units of arabic acid include D-galactose, L-rhamnose, L-arabinose and D-glucuronic acid. (1,3)-linked β-D-galactopyranosyl residues form the main chain which contains side chains branched by (1,6)-linkage. It is obtained from the sap of various acacias (Acacia senegal, Acacia syayl) and mimosa species by cutting into the bark of the trees and collecting the air-drying sap drops which are removed from the tree to close the wound.

Gum arabic is widely used in the food industry and particularly in the beverage industry to stabilize flavors and essential oils. From the nutrition physiology perspective, it is a ballast material of low energy value—for example half Me energy value of starch or maltodextrin. Accordingly, gum arabic is particularly suitable for the production of the sterol-containing powders according to the invention which are intended for use in beverages.

### Production

The powder-form sterol/stanol (ester) compositions according to the invention are obtainable by:
a) dispersing ground sterols and/or stanols, and/or esters thereof, in water heated to about 80 to 100° C. by intensive stirring to form a dispersion,
b) adding a natural macromolecular colloid former selected from the group consisting of gum arabic, gelatine, tragacanth, an alginate, carrageen, guar gum and xanthan, and mixtures thereof, preferably gum arabic as a dry powder, to the dispersion in a quantity ratio of a) to b) of about 10:90 to 80:20,
c) homogenizing the dispersion under high pressure; and
d) spray-drying the homogenized dispersion.

Micronized sterols and stanols with a mean particle size of about 0.01 to 50 μm, preferably about 0.05 to 30 μm and more particularly about 0.1 to 10 μm are preferably used in step a). Gum arabic is preferably used as the natural macromolecular colloid former.

Step d) can eliminate the need for emulsifiers because the spray drying process results in coating of the sterols and/or stanols poorly dispersible in aqueous systems with the carrier materials. Thus incorporation of the compositions of the invention, particularly in aqueous systems, is significantly improved by the resulting hydrophilic surface.

Accordingly, the present invention also relates to a process for the production of sterol-containing powders which process comprises:
a) dispersing micronized sterols and/or stanols in water heated to about 80 to 100° C. by intensive stirring,
b) adding a natural macromolecular colloid former selected from the group consisting of gum arabic, gelatine, tragacanth, an alginate, carrageen, guar gum and xanthan, and mixtures thereof, preferably gum arabic as a dry powder, to the dispersion in a ratio of a) to b) of about 10:90 to 80:20,
c) homogenizing the dispersion under high pressure; and
d) spray-drying the homogenized dispersion.

### Particle Size

The particle size distribution of the composition according to the invention is dependent upon the production process. In the preferred spray drying process, the mean particle size is generally between about 0.01 and 50 μm, preferably between about 0.1 and 30 μm and, more particularly in the range from about 1 to 10 μm.

The particle size distribution was determined with a Beckman Coulter LS 230 using the optical model emulsion.rfd PIDS included (of 14.08.01) in accordance with the 1994 operating instructions. Water was used as the measuring medium. The particle size measurements were carried out immediately after preparation of the dispersions. Selected dispersions were storage-tested (see Examples).

The following examples are illustrative of the invention and should not be construed in any manner as limiting of the scope of the present invention.

### EXAMPLES

1650 g water were heated to about 90° C. 200 g Generol 122 NG (ground sterol from Cognis Deutschland GmbH & Co. KG) were then added with intensive stirring, stirring being continued until the sterol was finely dispersed. 319 grams of gum arabic (dry matter 94%) were then added. The mixture was cooled to 75° C. and the dispersion was homogenized (at 220130 bar) in a Schröder LAB 100/60 homogenizer and spray dried (APV Anhydro type 3S).

Spray drying conditions:

| | | |
|---|---|---|
| entry temperature: | 185° | C. |
| exit temperature: | 93° | C. |
| atomizer: | 24000 | rpm |

The powder obtained could readily be stirred into water, milk and orange juice at room temperature and, in a concentration of 1% by weight, based on the sterol-containing beverage, did not have an unpleasant taste either during or after drinking. Measurement of the particle size distribution of the powder by laser diffractometry (Beckman Coulter LS 230) produced a d50 value of 5 μm and a d90 value of 29 μm.

The test was repeated with the following compositions:

powder 1: 40% soya sterol and 60% gum arabic
powder 2: 40% tall oil/rape sterol (80:20) and 60% gum arabic
powder 3: 40% tall oil/rape sterol and 30% gum arabic and 30% maltodextrin
powder 4: 40% tall oil/rapeseed sterol and 60% maltodextrin
Dispersion in the following systems with stirring at 600 rpm was carried out (0.4% and 1% sterol content):

| | Powder 1 | Powder 2 | Powder 3 | Powder 4 |
|---|---|---|---|---|
| Water 15° C. | Readily stirrable | Readily stirrable | Stirrable | Poorly stirrable |
| Water 60° C. | Readily stirrable | Readily stirrable | Readily stirrable | Poorly stirrable |
| Milk 18° C. | Readily stirrable | Readily stirrable | Readily stirrable | Stirrable |
| O juice | Readily stirrable | Readily stirrable | Stirrable | Poorly stirrable |

Sensory evaluation showed that powders 1 and 2 tasted neutral in water and that both milk and orange juice were evaluated as very good. No typical sterol taste could be detected. A clear difference was observed in relation to the sterol dispersion with no colloid former and powders 3 and 4, which produced a relatively poor sensory evaluation.

What we claim is:

1-10. (canceled)

11. A composition in powder form which comprises:
(a) a sterol and/or stanol, and/or esters thereof, and
(b) a natural macromolecular colloid former selected from the group consisting of gum arabic, gelatine, tragacanth, an alginate, carrageen, guar gum, xanthan and mixtures thereof, in a ratio of a) to b) of about 10:90 to 80:20.

12. The composition of claim 11 wherein component (a) is an unesterified sterol and/or stanol.

13. The composition of claim 11 wherein component (b) is gum arabic.

14. The composition of claim 11 which is emulsifier-free.

15. The composition of claim 11 which has a mean particle size of about 0.01 to 50 μm.

16. The composition of claim 11 which comprises
a) about 30 to 50% by weight of a sterol and/or stanol, and/or esters thereof and;
b) about 50 to 70% by weight gum arabic, based on the total weight of the composition.

17. A food product which comprises about 0.1 to 50% by weight of the composition of claim 11 based on the total weight of the food product.

18. The food product of claim 17 which is a beverage or milk product.

19. A process for the production of sterol-containing powders which comprises:
a) dispersing a micronized sterol and/or stanol, and/or esters thereof, in water heated to about 80 to 100° C. by stirring to form a dispersion,
b) adding a natural macromolecular colloid former selected from the group consisting of gum arabic, gelatine, tragacanth, an alginate, carrageen, guar gum, xanthan, and mixtures thereof, to the dispersion in a ratio of a) to b) of about 10:90 to 80:20,
c) homogenizing the dispersion under high pressure; and
d) spray-drying the homogenized dispersion.

20. The process of claim 19 wherein component (b) is added in dry powder form.

21. The process of claim 19 wherein component (b) is gum arabic.

22. Sterol-containing powders made by the process of:
a) dispersing ground sterols and/or stanols and/or esters thereof in water heated to about 80 to 100° C. by stirring to form a dispersion,
b) adding gum arabic as a dry powder to the dispersion,
c) homogenizing the dispersion under high pressure; and
d) spray-drying the homogenized dispersion.
